(19) United States
(12) Patent Application Publication
(10) Pub. No.: US 2024/0160183 A1
(43) Pub. Date: May 16, 2024

(54) CONTEXT-AWARE AUGMENTATION OF DIGITAL THREE-DIMENSIONAL MODEL

(71) Applicant: INTERNATIONAL BUSINESS MACHINES CORPORATION, ARMONK, NY (US)

(72) Inventors: Saraswathi Sailaja Perumalla, Visakhapatnam (IN); Sarbajit K. Rakshit, Kolkata (IN); Tushar Agrawal, West Fargo, ND (US); Raghupatruni Nagesh, Visakhapatnam (IN)

(21) Appl. No.: 18/054,987

(22) Filed: Nov. 14, 2022

(51) Int. Cl. G05B 19/4099; B29C 64/393; B33Y 50/02; C12M 1/00; C12M 1/36; C12M 3/06

(52) U.S. Cl. CPC G05B 19/4099; B29C 64/393; B33Y 50/02; C12M 21/02; C12M 23/16; C12M 41/48; C12M 43/08; G05B 2219/35134

(57) ABSTRACT

A computer-implemented method for augmentation and printing of a three-dimensional (3D) object is provided. The computer-implemented method includes analyzing a model of the 3D object, information of sensors for deployment on the 3D object and environmental parameters of a location where the 3D object is deployable and determining, from results of the analyzing, a surface contour of the 3D object, power requirements of the sensors and power levels that can be generated at the location by algae-based power generation. The computer-implemented method further includes augmenting the model with microfluidic circuitry models for supporting the algae-based power generation on the surface contour to meet the power requirements to an extent possible given the power levels, printing the 3D object and microfluidic circuitry according to the model and the microfluidic circuitry models and supplying the microfluidic circuitry with algae for the algae-based power generation.

### BACKGROUND

The present invention generally relates to artificial intelligence (AI), and more specifically, to a method and system for context-aware augmentation of a digital three-dimensional (3D) model to include a microfluidic circuit for blue-algae power generation.

3D printing or additive manufacturing is a process of making solid objects from a digital file. The digital file includes instructions that are provided to a 3D printer to control the printer in the creation of a 3D printed object. 3D printers utilize various forms of additive fabrication processes. In an additive fabrication process, an object is created by laying down successive layers of material until the object is created. Each of these layers can be seen as a thinly sliced cross-section of the object. 3D printing enables the production of complex shapes using less material than traditional manufacturing methods.

### SUMMARY

Embodiments of the present invention are directed to a computer-implemented method for augmentation and printing of a three-dimensional (3D) object. A non-limiting example of the computer-implemented method includes analyzing a model of the 3D object, information of sensors for deployment on the 3D object and environmental parameters of a location where the 3D object is deployable and determining, from results of the analyzing, a surface contour of the 3D object, power requirements of the sensors and power levels that can be generated at the location by algae-based power generation. The computer-implemented method further includes augmenting the model with microfluidic circuitry models for supporting the algae-based power generation on the surface contour to meet the power requirements to an extent possible given the power levels, printing the 3D object and microfluidic circuitry according to the model and the microfluidic circuitry models and supplying the microfluidic circuitry with algae for the algae-based power generation.

Embodiments of the invention further provide computer program products and computer systems having substantially the same features and technical benefits as the above-described computer-implemented methods.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 is a block diagram of components of a machine learning training and inference system according to one or more embodiments of the present invention;

FIG. 2 is a flow diagram illustrating a computer-implemented method of augmenting and printing a three-dimensional (3D) object in accordance with one or more embodiments of the present invention;

FIG. 3 is a flow diagram illustrating further details of the computer-implemented method of augmenting and printing a 3D object of FIG. 2 in accordance with one or more embodiments of the present invention;

FIG. 4 is a graphical illustrating of operations of the methods of FIGS. 2 and 3 in accordance with one or more embodiments of the present invention; and

FIG. 5 is a schematic diagram of a computing environment for implementing one or more embodiments of the present invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the disclosed embodiments, the various elements illustrated in the figures are provided with two or three digit reference numbers. With minor exceptions, the leftmost digit(s) of each reference number correspond to the figure in which its element is first illustrated.

### DETAILED DESCRIPTION

Turning now to an overview of technologies that are more specifically relevant to aspects of the invention, 3D printing is being used for manufacturing 3D objects and can also be used for repairing 3D objects. In some cases, 3D printing systems can use the capabilities of autonomous robotic systems to effectively collaborate with a swarm of 3D printing robots to perform printing in a collaborative manner.

In addition to advances in 3D printing, research into the potential of algae as a fuel source is moving quickly.

Using current 3D printing methods, a complete mechanism or machine can be manufactured, with different moving components and an appropriate number of sensors on different parts. Algae-based power generation can be provided for powering those sensors. Thus, a need exists for a method and system by which each and every sensor on a 3D printed mechanism or machine is powered by algae-based power generation while, at the same time, providing maintainability of the algae-based power generation.

Turning now to an overview of the aspects of the invention, one or more embodiments of the invention address shortcomings of the above-described approach by providing context-aware augmentation of 3D printing files to include microfluidic circuitry in which algae-based power generation is made possible for powering various sensors.

The above-described aspects of the invention address the shortcomings of known approaches by providing for an analysis of a shape and dimensions of a 3D object represented by a 3D digital model (i.e., an SLT file), an analysis of numbers and types of sensors and their relative positions on the 3D object and an estimation of power requirements of the sensors as well as an availability of carbon dioxide at a location of the 3D object. Through artificial intelligence (AI), the 3D digital model will then be augmented with appropriate types of microfluidic circuitry in which algae (i.e., blue algae) can be charged and recharged to provide required power to the sensors. The augmentation will specify microfluidic circuitry passage diameters and lengths, selections of appropriate materials, such as transparent materials, to allow the algae to generate power, selections of hybrid power supplies, such as batteries and grid power, to supplement the algae-based power generation and selections of various microfluidic circuitry arrangements and formations.

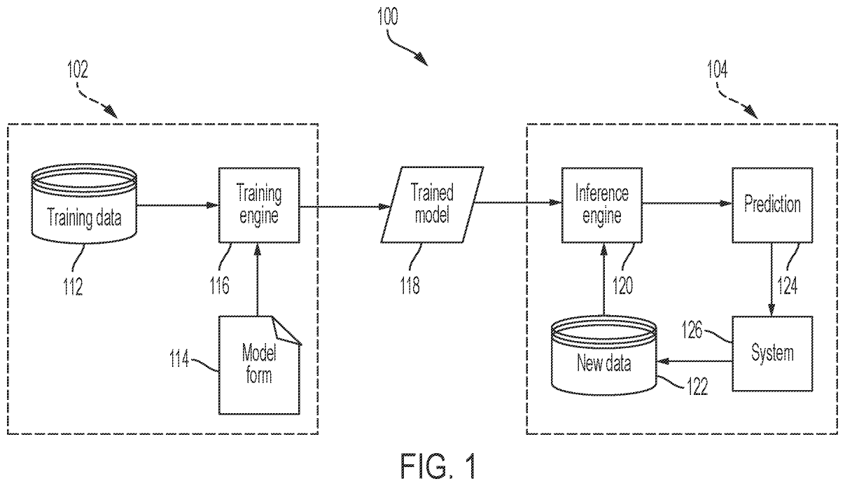

Turning now to a more detailed description of aspects of the present invention, FIG. 1 depicts a block diagram of components of a machine learning training and inference system 100. The machine learning training and inference system 100, in accordance with one or more embodiments of the invention, can utilize machine learning techniques to perform tasks, such as augmentation and printing of a 3D object. More specifically, one or more embodiments of the invention described herein can incorporate and utilize rule-based decision making and artificial intelligence (AI) reasoning to accomplish the various operations described herein, namely analyzing a model of the 3D object, information of sensors for deployment on the 3D object and environmental parameters of a location where the 3D object is deployable, determining, from results of the analyzing, a surface contour of the 3D object, power requirements of the sensors and power levels that can be generated at the location by algae-based power generation, augmenting the model with microfluidic circuitry models for supporting the algae-based power generation on the surface contour to meet the power requirements to an extent possible given the power levels, printing the 3D object and microfluidic circuitry according to the model and the microfluidic circuitry models and supplying the microfluidic circuitry with algae for the algae-based power generation.

Embodiments of the invention utilize AI, which includes a variety of so-called machine learning technologies. The phrase “machine learning” broadly describes a function of electronic systems that learn from data. A machine learning system, engine, or module can include a trainable machine learning algorithm that can be trained, such as in an external cloud environment, to learn functional relationships between inputs and outputs, and the resulting model (sometimes referred to as a “trained neural network,” “trained model,” and/or “trained machine learning model”) can be used for managing information during a web conference, for example. In one or more embodiments of the invention, machine learning functionality can be implemented using an artificial neural network (ANN) having the capability to be trained to perform a function. In machine learning and cognitive science, ANNs are a family of statistical learning models inspired by the biological neural networks of animals, and in particular the brain. ANNs can be used to estimate or approximate systems and functions that depend on a large number of inputs. Convolutional neural networks (CNN) are a class of deep, feed-forward ANNs that are particularly useful at tasks such as, but not limited to analyzing visual imagery and natural language processing (NLP). Recurrent neural networks (RNN) are another class of deep, feed-forward ANNs and are particularly useful at tasks such as, but not limited to, unsegmented connected handwriting recognition and speech recognition. Other types of neural networks are also known and can be used in accordance with one or more embodiments of the invention described herein.

ANNs can be embodied as so-called “neuromorphic” systems of interconnected processor elements that act as simulated “neurons” and exchange “messages” between each other in the form of electronic signals. Similar to the so-called “plasticity” of synaptic neurotransmitter connections that carry messages between biological neurons, the connections in ANNs that carry electronic messages between simulated neurons are provided with numeric weights that correspond to the strength or weakness of a given connection. The weights can be adjusted and tuned based on experience, making ANNs adaptive to inputs and capable of learning. For example, an ANN for handwriting recognition is defined by a set of input neurons that can be activated by the pixels of an input image. After being weighted and transformed by a function determined by the network's designer, the activation of these input neurons are then passed to other downstream neurons, which are often referred to as “hidden” neurons. This process is repeated until an output neuron is activated. The activated output neuron determines which character was input. It should be appreciated that these same techniques can be applied in the case of localizing a target object referred by a compositional expression from an image set with similar visual elements as described herein.

The machine learning training and inference system 100 performs training 102 and inference 104. During training 102, a training engine 116 trains a model (e.g., the trained model 118) to perform a task. Inference 104 is the process of implementing the trained model 118 to perform the task in the context of a larger system (e.g., a system 126).

The training 102 begins with training data 112, which can be structured or unstructured data. The training engine 116 receives the training data 112 and a model form 114. The model form 114 represents a base model that is untrained. The model form 114 can have preset weights and biases, which can be adjusted during training. It should be appreciated that the model form 114 can be selected from many different model forms depending on the task to be performed. For example, where the training 102 is to train a model to perform image classification, the model form 114 can be a model form of a CNN (convolutional neural network). The training 102 can be supervised learning, semi-supervised learning, unsupervised learning, reinforcement learning, and/or the like, including combinations and/or multiples thereof. For example, supervised learning can be used to train a machine learning model to classify an object of interest in an image. To do this, the training data 112 includes labeled images, including images of the object of interest with associated labels (ground truth) and other images that do not include the object of interest with associated labels. In this example, the training engine 116 takes as input a training image from the training data 112, makes a prediction for classifying the image, and compares the prediction to the known label. The training engine 116 then adjusts weights and/or biases of the model based on results of the comparison, such as by using backpropagation. The training 102 can be performed multiple times (referred to as “epochs”) until a suitable model is trained (e.g., the trained model 118).

Once trained, the trained model 118 can be used to perform inference 104 to perform a task. The inference engine 120 applies the trained model 118 to new data 122 (e.g., real-world, non-training data). For example, if the trained model 118 is trained to classify images of a particular object, such as a chair, the new data 122 can be an image of a chair that was not part of the training data 112. In this way, the new data 122 represents data to which the model 118 has not been exposed. The inference engine 120 makes a prediction 124 (e.g., a classification of an object in an image of the new data 122) and passes the prediction 124 to the system 126. The system 126 can, based on the prediction 124, taken an action, perform an operation, perform an analysis, and/or the like, including combinations and/or multiples thereof. In some embodiments of the invention, the system 126 can add to and/or modify the new data 122 based on the prediction 124.

In accordance with one or more embodiments of the invention, the predictions 124 generated by the inference engine 120 are periodically monitored and verified to ensure that the inference engine 120 is operating as expected. Based on the verification, additional training 102 can occur using the trained model 118 as the starting point. The additional training 102 can include all or a subset of the original training data 112 and/or new training data 112. In accordance with one or more embodiments of the invention, the training 102 includes updating the trained model 118 to account for changes in expected input data.

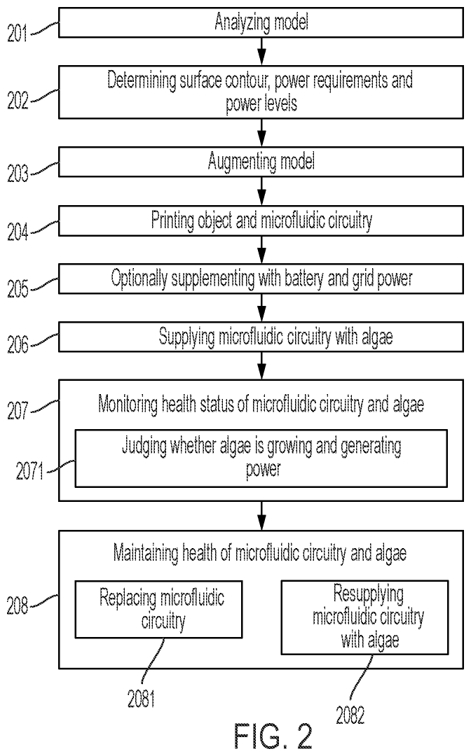

With reference to FIG. 2, a computer-implemented method 200 for augmentation and printing of a 3D object is provided. The computer-implemented method 200 includes, at block 201, analyzing a model of the 3D object (i.e., by analyzing a dimension, a shape and a size of the 3D object), analyzing information of sensors for deployment on the 3D object (i.e., by analyzing numbers, types and relative locations of the sensors) and analyzing environmental parameters of a location where the 3D object is deployable (i.e., by analyzing sunlight duration and direction, carbon dioxide concentrations and humidity). The computer-implemented method 200 also includes, at block 202 determining, from results of the analyzing, a surface contour of the 3D object from the analyzing of the model of the 3D object, power requirements of the sensors from the analyzing of the information of the sensors and power levels that can be generated at the location by algae-based power generation from the analyzing of the environmental parameters. The computer-implemented method further includes, at block 203, augmenting the model with microfluidic circuitry models for supporting the algae-based power generation on the surface contour to meet the power requirements to an extent possible given the power levels. The microfluidic circuitry models can include information that is representative of microfluidic circuit dimensions, shapes and sizes, microfluidic circuit materials especially where microfluidic circuit materials are transparent to allow light to penetrate to algae and microfluidic circuit positions on the surface contour of the 3D object. In addition, the computer-implemented method includes printing the 3D object and microfluidic circuitry according to the model and the microfluidic circuitry models at block 204 as well as optionally supplementing the microfluidic circuitry with at least one of battery and grid power at block 205 and supplying the microfluidic circuitry with algae for the algae-based power generation at block 206.

In accordance with further embodiments, the computer-implemented method 200 can also include monitoring a health status of the microfluidic circuitry and the algae at block 207 and taking action toward maintaining the health of the microfluidic circuitry and the algae at block 208. The monitoring of the health of the microfluidic circuitry and the algae can include judging whether the algae is growing and generating power at block 2071. The taking of the action toward maintaining the health of the microfluidic circuitry and the algae can include at least one of replacing the microfluidic circuitry at block 2081 and resupplying the microfluidic circuitry with the algae at block 2082.

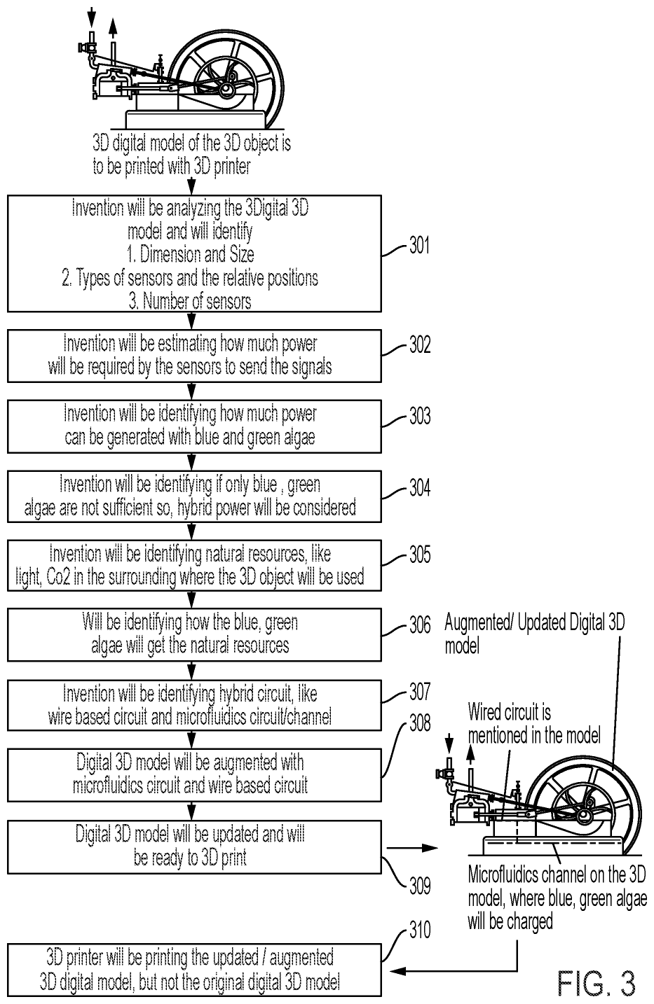

With reference to FIG. 3, further details of the computer-implemented method of FIG. 2 are illustrated. As shown in FIG. 3, an original digital 3D model of a 3D object that is to be printed is received. The original digital 3D model is then analyzed for dimension and size and for numbers and types of sensors at block 301. An estimate is developed for how much power will be required for the sensors at block 302 and a determination is made as to how much power can be generated with algae (blue and green) at block 303. A further determination is then made as to whether algae-based power generation needs to be supplemented at block 304. Next, natural resource availability will be identified at block 305, an ability of the algae to access the available natural resources will also be identified at block 306 and options for circuits (i.e., hybrid, wire-based, microfluidic, etc.) will be identified at block 307. At this point, the 3D digital model will be augmented and updated at blocks 308 and 309. The 3d digital model is then printed at block 310.

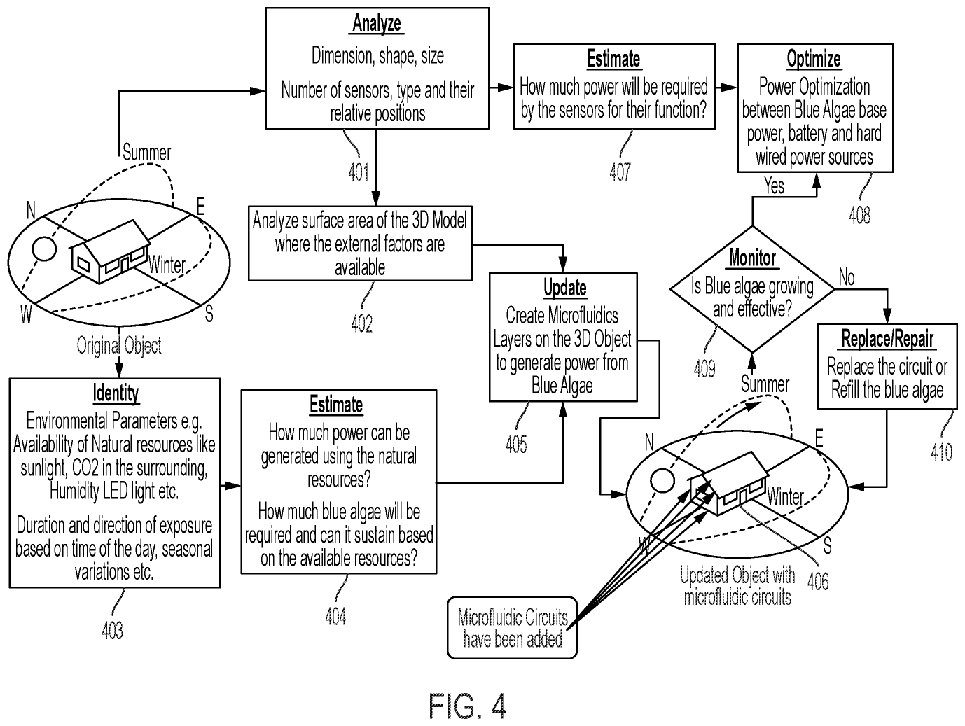

With reference to FIG. 4, a flow of the computer-implemented method 200 of FIG. 2 (and FIG. 3) will now be described. As shown in FIG. 4, a 3D digital model of a 3D object, such as a 3D printed house, is initially received. The 3D model is then analyzed for dimension and size and for numbers and types of sensors at block 401 so that a determination can be made as to a surface contour of the 3D object at block 402. At a same time, environmental parameters of a location where the 3D object is to be located are analyzed at block 403 to determine an availability of natural resources, such as sunlight and carbon dioxide, as well as a duration and a direction of light exposure. An estimate is then developed for how much power that can be generated with algae (blue and green) based on the available resources at block 404. The 3D digital model is then updated to include microfluidic circuitry for algae-based power generation at block 405 based on results of the analysis and the estimates. At this point, the 3D object and the microfluidic circuitry can be printed at block 406.

At block 407, an estimate as to power requirements of the sensors can be developed for an optimization operation at block 408. In this case, the optimization operation can be based on a monitoring of the algae at block 409, which can also be used to determine whether to replace/repair the microfluidic circuits or the algae at block 410.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

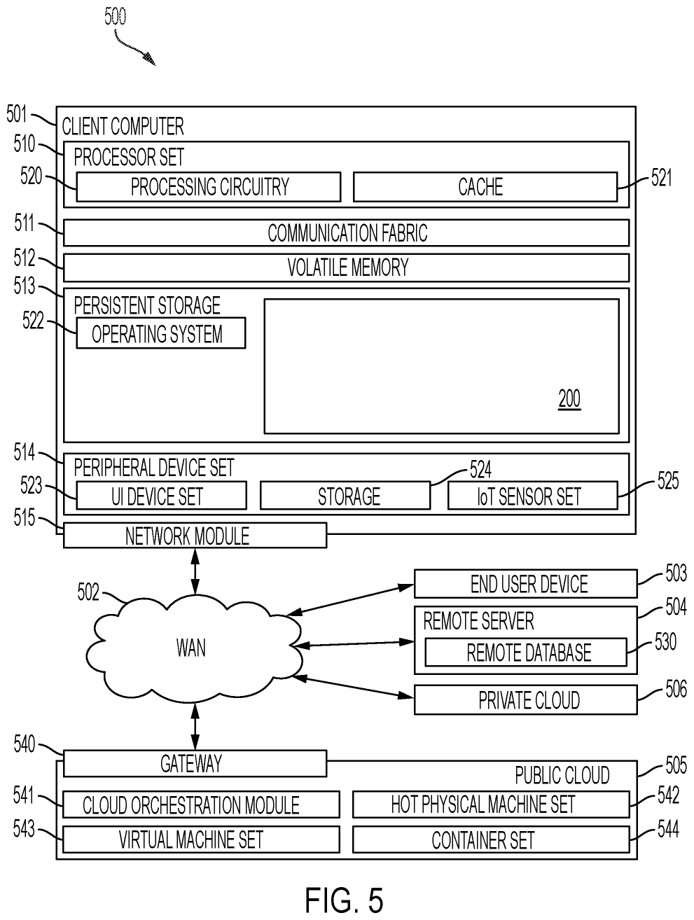

With reference to FIG. 5, a computer or computing device 500 that implements the computer-implemented method 200 of FIG. 2 (and FIG. 3) in accordance with one or more embodiments of the present invention is provided. The computing system of FIG. 5 contains an example of an environment for the execution of at least some of the computer code involved in performing the inventive methods, such as the block of the computer-implemented method 200 of FIG. 2 for managing information during a web conference. In addition to the computer-implemented method 200, the computing system 500 includes, for example, computer 501, wide area network (WAN) 502, end user device (EUD) 503, remote server 504, public cloud 505, and private cloud 506. In this embodiment, computer 501 includes processor set 510 (including processing circuitry 520 and cache 521), communication fabric 511, volatile memory 512, persistent storage 513 (including operating system 522 and the computer-implemented method 200, as identified above), peripheral device set 514 (including user interface (UI) device set 523, storage 524, and Internet of Things (IoT) sensor set 525), and network module 515. Remote server 504 includes remote database 530. Public cloud 505 includes gateway 540, cloud orchestration module 541, host physical machine set 542, virtual machine set 543, and container set 544.

The computer 501 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 530. As is well understood in the art of computer technology, and depending upon the technology, performance of a computer-implemented method may be distributed among multiple computers and/or between multiple locations. On the other hand, in this presentation of the computer-implemented method 200, detailed discussion is focused on a single computer, specifically computer 501, to keep the presentation as simple as possible. Computer 501 may be located in a cloud, even though it is not shown in a cloud in FIG. 5. On the other hand, computer 501 is not required to be in a cloud except to any extent as may be affirmatively indicated.

The processor set 510 includes one, or more, computer processors of any type now known or to be developed in the future. Processing circuitry 520 may be distributed over multiple packages, for example, multiple, coordinated integrated circuit chips. Processing circuitry 520 may implement multiple processor threads and/or multiple processor cores. Cache 521 is memory that is located in the processor chip package(s) and is typically used for data or code that should be available for rapid access by the threads or cores running on processor set 510. Cache memories are typically organized into multiple levels depending upon relative proximity to the processing circuitry. Alternatively, some, or all, of the cache for the processor set may be located “off chip.” In some computing environments, processor set 510 may be designed for working with qubits and performing quantum computing.

Computer readable program instructions are typically loaded onto computer 501 to cause a series of operational steps to be performed by processor set 510 of computer 501 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 521 and the other storage media discussed below. The program instructions, and associated data, are accessed by processor set 510 to control and direct performance of the inventive methods. In the computer-implemented method 200, at least some of the instructions for performing the inventive methods may be stored in the block of the computer-implemented method 200 in persistent storage 513.

Communication fabric 511 is the signal conduction path that allows the various components of computer 501 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

Volatile memory 512 is any type of volatile memory now known or to be developed in the future. Examples include dynamic type random access memory (RAM) or static type RAM. Typically, volatile memory 512 is characterized by random access, but this is not required unless affirmatively indicated. In computer 501, the volatile memory 512 is located in a single package and is internal to computer 501, but, alternatively or additionally, the volatile memory may be distributed over multiple packages and/or located externally with respect to computer 501.

Persistent storage 513 is any form of non-volatile storage for computers that is now known or to be developed in the future. The non-volatility of this storage means that the stored data is maintained regardless of whether power is being supplied to computer 501 and/or directly to persistent storage 513. Persistent storage 513 may be a read only memory (ROM), but typically at least a portion of the persistent storage allows writing of data, deletion of data and re-writing of data. Some familiar forms of persistent storage include magnetic disks and solid state storage devices. Operating system 522 may take several forms, such as various known proprietary operating systems or open source Portable Operating System Interface-type operating systems that employ a kernel. The code included in the block of the computer-implemented method 200 typically includes at least some of the computer code involved in performing the inventive methods.

Peripheral device set 514 includes the set of peripheral devices of computer 501. Data communication connections between the peripheral devices and the other components of computer 501 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 523 may include components such as a display screen, speaker, microphone, wearable devices (such as goggles and smart watches), keyboard, mouse, printer, touchpad, game controllers, and haptic devices. Storage 524 is external storage, such as an external hard drive, or insertable storage, such as an SD card. Storage 524 may be persistent and/or volatile. In some embodiments, storage 524 may take the form of a quantum computing storage device for storing data in the form of qubits. In embodiments where computer 501 is required to have a large amount of storage (for example, where computer 501 locally stores and manages a large database) then this storage may be provided by peripheral storage devices designed for storing very large amounts of data, such as a storage area network (SAN) that is shared by multiple, geographically distributed computers. IoT sensor set 525 is made up of sensors that can be used in Internet of Things applications. For example, one sensor may be a thermometer and another sensor may be a motion detector.

Network module 515 is the collection of computer software, hardware, and firmware that allows computer 501 to communicate with other computers through WAN 502. Network module 515 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 515 are performed on the same physical hardware device. In other embodiments (for example, embodiments that utilize software-defined networking (SDN)), the control functions and the forwarding functions of network module 515 are performed on physically separate devices, such that the control functions manage several different network hardware devices. Computer readable program instructions for performing the inventive methods can typically be downloaded to computer 501 from an external computer or external storage device through a network adapter card or network interface included in network module 515.

WAN 502 is any wide area network (for example, the internet) capable of communicating computer data over non-local distances by any technology for communicating computer data, now known or to be developed in the future. In some embodiments, the WAN 502 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device (EUD) 503 is any computer system that is used and controlled by an end user (for example, a customer of an enterprise that operates computer 501), and may take any of the forms discussed above in connection with computer 501. EUD 503 typically receives helpful and useful data from the operations of computer 501. For example, in a hypothetical case where computer 501 is designed to provide a recommendation to an end user, this recommendation would typically be communicated from network module 515 of computer 501 through WAN 502 to EUD 503. In this way, EUD 503 can display, or otherwise present, the recommendation to an end user. In some embodiments, EUD 503 may be a client device, such as thin client, heavy client, mainframe computer, desktop computer and so on.

Remote server 504 is any computer system that serves at least some data and/or functionality to computer 501. Remote server 504 may be controlled and used by the same entity that operates computer 501. Remote server 504 represents the machine(s) that collect and store helpful and useful data for use by other computers, such as computer 501. For example, in a hypothetical case where computer 501 is designed and programmed to provide a recommendation based on historical data, then this historical data may be provided to computer 501 from remote database 530 of remote server 504.

Public cloud 505 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 505 is performed by the computer hardware and/or software of cloud orchestration module 541. The computing resources provided by public cloud 505 are typically implemented by virtual computing environments that run on various computers making up the computers of host physical machine set 542, which is the universe of physical computers in and/or available to public cloud 505. The virtual computing environments (VCEs) typically take the form of virtual machines from virtual machine set 543 and/or containers from container set 544. It is understood that these VCEs may be stored as images and may be transferred among and between the various physical machine hosts, either as images or after instantiation of the VCE. Cloud orchestration module 541 manages the transfer and storage of images, deploys new instantiations of VCEs and manages active instantiations of VCE deployments. Gateway 540 is the collection of computer software, hardware, and firmware that allows public cloud 505 to communicate through WAN 502.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 506 is similar to public cloud 505, except that the computing resources are only available for use by a single enterprise. While private cloud 506 is depicted as being in communication with WAN 502, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 505 and private cloud 506 are both part of a larger hybrid cloud.

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method for augmentation and printing of a three-dimensional (3D) object, the computer-implemented method comprising:
analyzing, using a processor, a model of the 3D object, information of sensors for deployment on the 3D object and environmental parameters of a location where the 3D object is deployable;
responsive to results of the analyzing, determining a surface contour of the 3D object, power requirements of the sensors and power levels that can be generated at the location by algae-based power generation;
augmenting, using the processor, the model with microfluidic circuitry models for supporting the algae-based power generation on the surface contour to meet the power requirements to an extent possible given the power levels;
controlling a 3D printer to print the 3D object and microfluidic circuitry according to the model and the microfluidic circuitry models; and
supplying the microfluidic circuitry with algae for the algae-based power generation.

2. The computer-implemented method according to claim 1, wherein:
the analyzing of the model of the 3D object comprises analyzing a dimension, a shape and a size of the 3D object,
the analyzing of the information of the sensors for deployment on the 3D object comprises analyzing numbers, types and relative locations of the sensors, and
the analyzing of the environmental parameters of the location where the 3D object is deployable comprises analyzing sunlight duration and direction, carbon dioxide concentrations and humidity.

3. The computer-implemented method according to claim 1, wherein the microfluidic circuitry models comprise:
microfluidic circuit dimensions, shapes and sizes;
microfluidic circuit materials; and
microfluidic circuit positions on the surface contour of the 3D object.

4. The computer-implemented method according to claim 1, wherein the printing comprises supplementing the microfluidic circuitry with at least one of battery and grid power.

5. The computer-implemented method according to claim 1, further comprising:
monitoring health of the microfluidic circuitry and the algae; and
taking action toward maintaining the health of the microfluidic circuitry and the algae.

6. The computer-implemented method according to claim 1, wherein the monitoring of the health of the microfluidic circuitry and the algae comprises judging whether the algae is growing and generating power.

7. The computer-implemented method according to claim 1, wherein the taking of the action toward maintaining the health of the microfluidic circuitry and the algae comprises at least one of replacing the microfluidic circuitry and resupplying the microfluidic circuitry with the algae.

8. A computer program product for augmentation and printing of a three-dimensional (3D) object, the computer program product comprising one or more computer readable storage media having computer readable program code collectively stored on the one or more computer readable storage media, the computer readable program code being executed by a processor of a computer system to cause the computer system to perform a method comprising:
analyzing a model of the 3D object, information of sensors for deployment on the 3D object and environmental parameters of a location where the 3D object is deployable;
determining, from results of the analyzing, a surface contour of the 3D object, power requirements of the sensors and power levels that can be generated at the location by algae-based power generation;
augmenting the model with microfluidic circuitry models for supporting the algae-based power generation on the surface contour to meet the power requirements to an extent possible given the power levels;
printing the 3D object and microfluidic circuitry according to the model and the microfluidic circuitry models; and
supplying the microfluidic circuitry with algae for the algae-based power generation.

9. The computer program product according to claim 8, wherein:
the analyzing of the model of the 3D object comprises analyzing a dimension, a shape and a size of the 3D object,
the analyzing of the information of the sensors for deployment on the 3D object comprises analyzing numbers, types and relative locations of the sensors, and
the analyzing of the environmental parameters of the location where the 3D object is deployable comprises analyzing sunlight duration and direction, carbon dioxide concentrations and humidity.

10. The computer program product according to claim 8, wherein the microfluidic circuitry models comprise:
microfluidic circuit dimensions, shapes and sizes;
microfluidic circuit materials; and
microfluidic circuit positions on the surface contour of the 3D object.

11. The computer program product according to claim 8, wherein the printing comprises supplementing the microfluidic circuitry with at least one of battery and grid power.

12. The computer program product according to claim 8, wherein the method further comprises:
monitoring health of the microfluidic circuitry and the algae; and
taking action toward maintaining the health of the microfluidic circuitry and the algae.

13. The computer program product according to claim 8, wherein the monitoring of the health of the microfluidic circuitry and the algae comprises judging whether the algae is growing and generating power.

14. The computer program product according to claim 8, wherein the taking of the action toward maintaining the health of the microfluidic circuitry and the algae comprises at least one of replacing the microfluidic circuitry and resupplying the microfluidic circuitry with the algae.

15. A computing system comprising:
a processor;
a memory coupled to the processor; and
one or more computer readable storage media coupled to the processor, the one or more computer readable storage media collectively containing instructions that are executed by the processor via the memory to implement a method comprising:
analyzing a model of the 3D object, information of sensors for deployment on the 3D object and environmental parameters of a location where the 3D object is deployable;
determining, from results of the analyzing, a surface contour of the 3D object, power requirements of the sensors and power levels that can be generated at the location by algae-based power generation;
augmenting the model with microfluidic circuitry models for supporting the algae-based power generation on the surface contour to meet the power requirements to an extent possible given the power levels;
printing the 3D object and microfluidic circuitry according to the model and the microfluidic circuitry models; and
supplying the microfluidic circuitry with algae for the algae-based power generation.

16. The computing system according to claim 15, wherein:
the analyzing of the model of the 3D object comprises analyzing a dimension, a shape and a size of the 3D object,
the analyzing of the information of the sensors for deployment on the 3D object comprises analyzing numbers, types and relative locations of the sensors, and
the analyzing of the environmental parameters of the location where the 3D object is deployable comprises analyzing sunlight duration and direction, carbon dioxide concentrations and humidity.

17. The computing system according to claim 15, wherein the microfluidic circuitry models comprise:
microfluidic circuit dimensions, shapes and sizes;
microfluidic circuit materials; and
microfluidic circuit positions on the surface contour of the 3D object.

18. The computing system according to claim 15, wherein the printing comprises supplementing the microfluidic circuitry with at least one of battery and grid power.

19. The computing system according to claim 15, wherein the method further comprises:
monitoring health of the microfluidic circuitry and the algae; and
taking action toward maintaining the health of the microfluidic circuitry and the algae.

20. The computing system according to claim 15, wherein:
the monitoring of the health of the microfluidic circuitry and the algae comprises judging whether the algae is growing and generating power, and
the taking of the action toward maintaining the health of the microfluidic circuitry and the algae comprises at least one of replacing the microfluidic circuitry and resupplying the microfluidic circuitry with the algae.
